(19) United States
(12) Patent Application Publication
(10) Pub. No.: US 2018/0000334 A1
(43) Pub. Date: Jan. 4, 2018

(54) BIOLOGICAL OBSERVATION APPARATUS

(71) Applicant: OLYMPUS CORPORATION, Tokyo (JP)

(72) Inventor: Koki MORISHITA, Tokyo (JP)

(73) Assignee: OLYMPUS CORPORATION, Tokyo (JP)

(21) Appl. No.: 15/704,415

(22) Filed: Sep. 14, 2017

Related U.S. Application Data
(63) Continuation of application No. PCT/JP2015/058459, filed on Mar. 20, 2015.

(51) Int. Cl. A61B 1/06; A61B 1/04; A61B 1/00

(52) U.S. Cl. CPC A61B 1/0638; A61B 1/0684; A61B 1/00009; A61B 1/00045; A61B 1/04

(57) ABSTRACT

Provided is a biological observation apparatus including: illuminating portions that irradiate biological tissue with illumination light including light in R, G, and B regions, respectively; an image acquisition portion that acquires image signals from reflected light of the illumination light coming from the biological tissue; narrow-band-light generating portions that are disposed in the illuminating portions or the image acquisition portion and that, in wavelength bands of the illumination light, generate two narrow-band beams for at least one of the R, G, and B wavelength bands constituting the illumination light, on either side of a central wavelength of that wavelength band; and an image-generating portion that generates an image on the basis of two or more types of the image signals obtained from the reflected light including two or more narrow bands acquired by the image acquisition portion.

### CROSS-REFERENCE TO RELATED APPLICATIONS

This is a continuation of International Application PCT/JP2015/058459, with an international filing date of Mar. 20, 2015, which is hereby incorporated by reference herein in its entirety.

### TECHNICAL FIELD

The present invention relates to a biological observation apparatus.

### BACKGROUND ART

In the related art, there are known endoscope systems with which special-light observation is performed by using narrow-band light (for example, see Patent Literatures 1 and 2).

With the endoscope system of Patent Literature 1, it is possible to perform observation by switching between normal-light observation and narrow-band-light observation in which blood (blood vessels) can be emphasized.

In addition, with Patent Literature 2, it is possible to display an appropriate emphasized image in accordance with the observation subject by performing observation with a plurality of wavelength sets by using a spectral estimation method (pseudo-narrow-band observation).

### CITATION LIST

### Patent Literature

{PTL 1} Japanese Unexamined Patent Application, Publication No. 2006-68113

{PTL 2} Japanese Unexamined Patent Application, Publication No. 2011-194082

### SUMMARY OF INVENTION

An object of the present invention is to provide a biological observation apparatus with which it is possible to perform multiple types of special-light observation in the visible-light region by using a simple configuration.

### SOLUTION TO PROBLEM

An aspect of the present invention is a biological observation apparatus including: an illuminating portion that irradiates biological tissue with illumination light including light in R, G, and B regions, respectively; an image acquisition portion that acquires image signals from reflected light of the illumination light coming from the biological tissue; a narrow-band-light generating portion that is disposed in the illuminating portion or the image acquisition portion and that, in wavelength bands of the illumination light, generates two narrow-band beams for at least one of the R, G, and B wavelength bands constituting the illumination light, on either side of a central wavelength of that wavelength band; and an image-generating portion that generates an image on the basis of two or more types of the image signals obtained from the reflected light including two or more narrow bands acquired by the image acquisition portion.

Another aspect of the present invention is a biological observation apparatus including: an illuminating portion that irradiates biological tissue with illumination light including light in R, G, and B regions, respectively; an image acquisition portion that acquires image signals from reflected light of the illumination light coming from the biological tissue; a narrow-band-light generating portion that is disposed in the illuminating portion or the image acquisition portion and that, in the wavelength band of the illumination light, generates light in a first narrow band including a wavelength at which absorption characteristics of an observation subject component reach a maximum and light in a second narrow band that is different from the first narrow band for at least one of the R, G, and B wavelength bands constituting the illumination light; and an image-generating portion that generates an image on the basis of two or more types of the image signals obtained from the reflected light including two or more narrow bands acquired by the image acquisition portion.

In the above-described aspect, the observation subject component may be p-carotene or hemoglobin.

In addition, in the above-described aspect, the image-generating portion may generate a plurality of images including a normal observation image in which the image signals acquired by the image acquisition portion, which are obtained from the reflected light including all narrow bands generated by the narrow-band-light generating portion, are used in combinations and a display portion that simultaneously displays the plurality of images including the normal observation image may be provided.

### BRIEF DESCRIPTION OF DRAWINGS

FIG. 1 is an overall configuration diagram showing a biological observation apparatus according to a first embodiment of the present invention.

FIG. 2 is a front view of a filter turret of the biological observation apparatus in FIG. 1.

FIG. 3A is a diagram showing sensitivity characteristics of a color CCD provided in an image acquisition portion of the biological observation apparatus in FIG. 1.

FIG. 3B is a diagram showing transmittance characteristics of a first spectral filter provided in a light-source portion of the biological observation apparatus in FIG. 1.

FIG. 3C is a diagram showing transmittance characteristics of a second spectral filter provided in the light-source portion of the biological observation apparatus in FIG. 1.

FIG. 4 is an overall configuration diagram showing a biological observation apparatus according to a second embodiment of the present invention.

FIG. 5A is a diagram showing transmittance characteristics of a first spectral filter provided in a light-source portion of the biological observation apparatus in FIG. 4.

FIG. 5B is a diagram showing transmittance characteristics of a second spectral filter provided in the light-source portion of the biological observation apparatus in FIG. 4.

FIG. 6A is a diagram showing absorption characteristics of hemoglobin contained in biological tissue.

FIG. 6B is a diagram showing absorption characteristics of β-carotene contained in the biological tissue.

FIG. 6C is a diagram showing absorption characteristics of methylene blue, which is one of the exogenous dyes administered to the biological tissue.

FIG. 7A is a diagram showing transmittance characteristics of a first spectral filter in the case in which hemoglobin is used as an observation subject component in a biological observation apparatus according to a third embodiment of the present invention.

FIG. 7B is a diagram showing transmittance characteristics of a second spectral filter in the case in which hemoglobin is used as the observation subject component in the biological observation apparatus according to the third embodiment of the present invention.

FIG. 8A is a diagram showing transmittance characteristics of a first spectral filter according to another modification of the biological observation apparatus in FIG. 4.

FIG. 8B is a diagram showing transmittance characteristics of a second spectral filter according to another modification of the biological observation apparatus in FIG. 4.

FIG. 9 is an overall configuration diagram showing a modification in which a six-color LED is used as the light-source portion of the biological observation apparatus in FIG. 4.

FIG. 10 is a diagram showing wavelength characteristics of the intensity of the six-color LED in FIG. 9.

FIG. 11A is a diagram showing another modification of the biological observation apparatus in FIG. 4 and transmittance characteristics of a first spectral filter thereof when observing the oxygen saturation level.

FIG. 11B is a diagram showing another modification of the biological observation apparatus in FIG. 4 and showing transmittance characteristics of a second spectral filter thereof when observing the oxygen saturation level.

FIG. 12 is an overall configuration diagram showing another modification of the biological observation apparatus in FIG. 4 and a case in which a beam splitter is disposed in the image acquisition portion thereof.

FIG. 13 is a diagram showing reflectance characteristics of the beam splitter of the biological observation apparatus in FIG. 12.

### DESCRIPTION OF EMBODIMENTS

A biological observation apparatus 1 according to a first embodiment of the present invention will be described below with reference to the drawings.

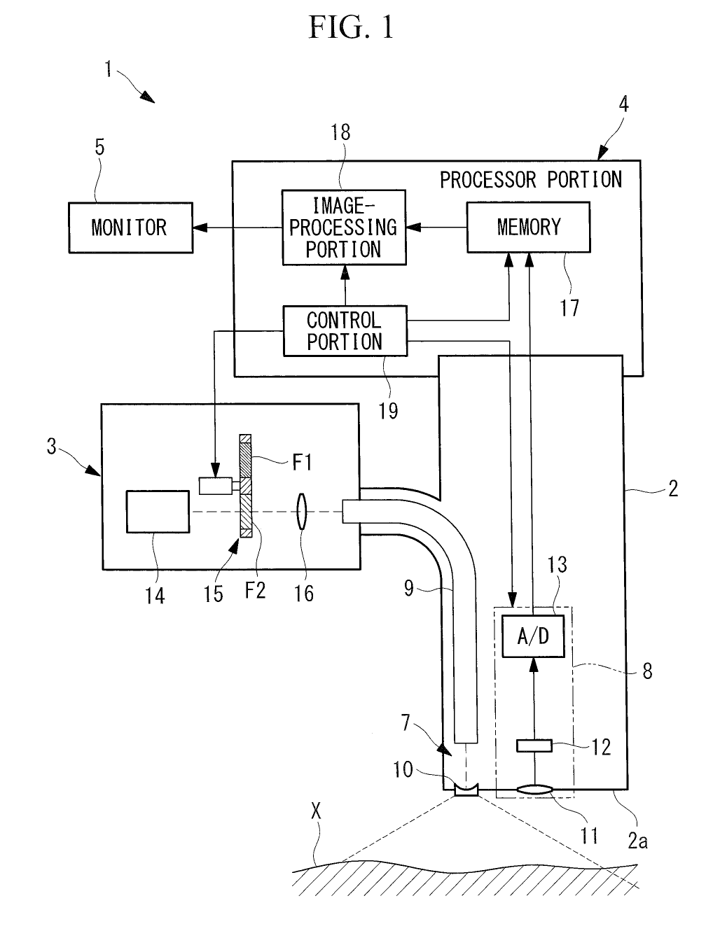

As shown in FIG. 1, the biological observation apparatus 1 according to this embodiment is an endoscope apparatus provided with: an inserted portion 2 that is inserted into a living organism; a light-source portion 3 that is connected to the inserted portion 2; a processor portion 4 that is connected to the inserted portion 2; and a monitor (display portion) 5 that displays an image generated by the processor portion 4.

The inserted portion 2 is provided with an illumination optical system 7 that irradiates an imaging subject with light input from the light-source portion 3 and an imaging optical system (image acquisition portion) 8 that captures reflected light coming from the imaging subject. The illumination optical system 7 is provided with a light-guide cable 9 that is disposed over the entire length of the inserted portion 2 and that guides, to a distal end 2a, the light that has entered from the light-source portion 3 on the basal-end side and a spreading optical system 10 that radiates the light guided by the light-guide cable 9 in the forward direction from the distal end 2a of the inserted portion 2. The light-source portion 3 and the illumination optical system 7 constitute an illuminating portion.

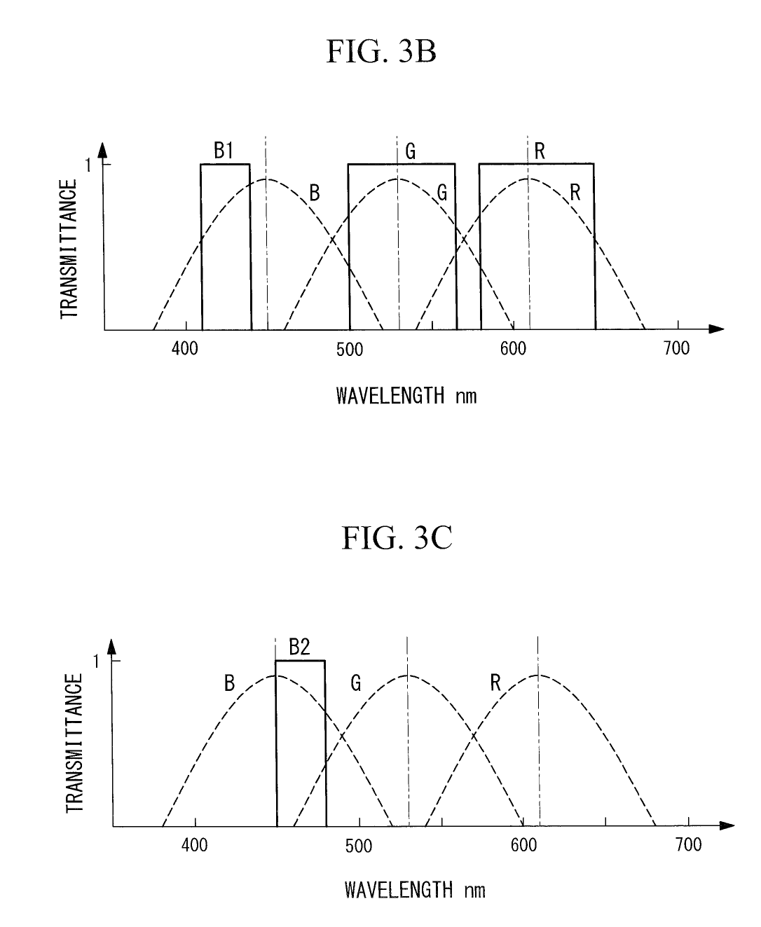

The imaging optical system 8 is provided with: a lens 11 that forms, in an image-acquisition device 12, an image of reflected light coming from biological tissue X irradiated with light from the illumination optical system 7; and the image-acquisition device 12 that captures the light focused by the lens 11. In the figures, reference sign 13 is an A/D converter that converts image signals acquired by the image-acquisition device 12 to digital signals. The image-acquisition device 12 is a color CCD in which filters that transmit blue, green, and red light are provided in individual pixels. The sensitivity characteristics of the image-acquisition device 12 are as shown in FIG. 3A.

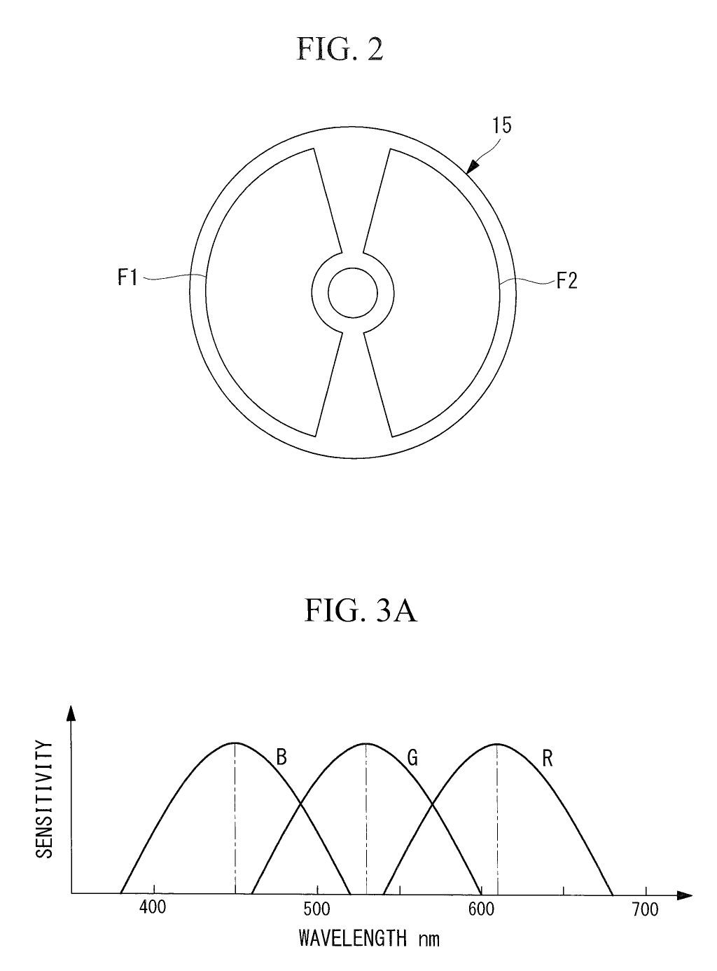

As shown in FIGS. 1 and 2, the light-source portion 3 is provided with: a xenon lamp 14 that generates white light; a filter turret 15 provided with two spectral filters F1 and F2 that extract two sets of narrow-band light from the white light emitted from the xenon lamp 14; and a focusing lens 16 that makes the narrow-band light extracted by the filter turret 15 enter the light-guide cable 9. The two spectral filters F1 and F2 are three-band filters individually having three transmission wavelength bands.

As shown in FIG. 3B, the first spectral filter F1 has B1 (410 to 440 nm), G (500 to 570 nm), and R (580 to 650 nm) transmission wavelength bands. In the figure, the broken line indicates the sensitivity of the color CCD 12, and the dashed lines individually indicate the central wavelengths of the R, G, and B wavelength bands.

As shown in FIG. 3C, the second spectral filter F2 has a B2 (450 to 480 nm) transmission wavelength band. The G and R transmission wavelength bands thereof are the same as those of the first spectral filter F1. In the figure, the broken line indicates the sensitivity of the color CCD 12, and the dashed lines individually indicate the central wavelengths of the R, G, and B wavelength bands.

When the respective spectral filters F1 and F2 are disposed in the optical path, the wavelength characteristics of light individually captured at the R, G, and B pixels of the color CCD 12 differ at the B pixels. Thus, by using combinations of the two types of spectral filters F1 and F2 and the three types of pixels, that is, the R, G, and B pixels, it is possible to obtain image signals having different wavelength components. In other words, the two spectral filters F1 and F2 constitute a narrow-band-light generating portion that extracts, from the light in the B wavelength band constituting the illumination light, two narrow-band beams on either side of the central wavelength of the wavelength band.

As shown in FIG. 1, the processor portion 4 is provided with: a memory 17 that stores the image signals acquired by the image-acquisition device 12; an image-processing portion (image-generating portion) 18 that processes the image signals stored in the memory 17; and a control portion 19 that controls the light-source portion 3, the image-acquisition device 12, the memory 17, and the image-processing portion 18.

The image-processing portion 18 is configured so as to generate images shown in Table 1 by using combinations of the image signals corresponding to the individual wavelengths in Table 1 stored in the memory 17.

TABLE 1

| | B | G | R |
|---|---|---|---|
| NORMAL OBSERVATION IMAGE | B1 + B2 | G | R |
| SURFACE-LAYER OBSERVATION IMAGE | B1 | G | R |
| DEEP-LAYER OBSERVATION IMAGE | B2 | G | R |

The individual images generated by the image-processing portion 18 will be described below in detail.

A normal observation image is an image that is constituted of all image signals of the R, G, B1, and B2 wavelength bands acquired by the image-acquisition device 12 (among R, G, and B image signals constituting the color image, wherein image signals in which B1 and B2 image signals are added are used as B image signals, and R and G image signals are used without modification). Because the image signals in all of the R, G, and B regions are composed of signals individually containing nearly all wavelength components, it is possible to obtain, in all of the R, G, and B wavelength bands, image signals that are close to those obtained in a state in which light including all of the respective R, G, and B wavelength bands is radiated. In other words, it is possible to generate a normal observation image in which colors close to those of an image obtained during white-light illumination are reproduced.

A surface-layer observation image is a special-light image that is constituted of the image signals of R, G, and B1 wavelength bands acquired by the image-acquisition device 12.

A deep-layer observation image is a special-light image that is constituted of the image signals of R, G, and B2 wavelength bands acquired by the image-acquisition device 12.

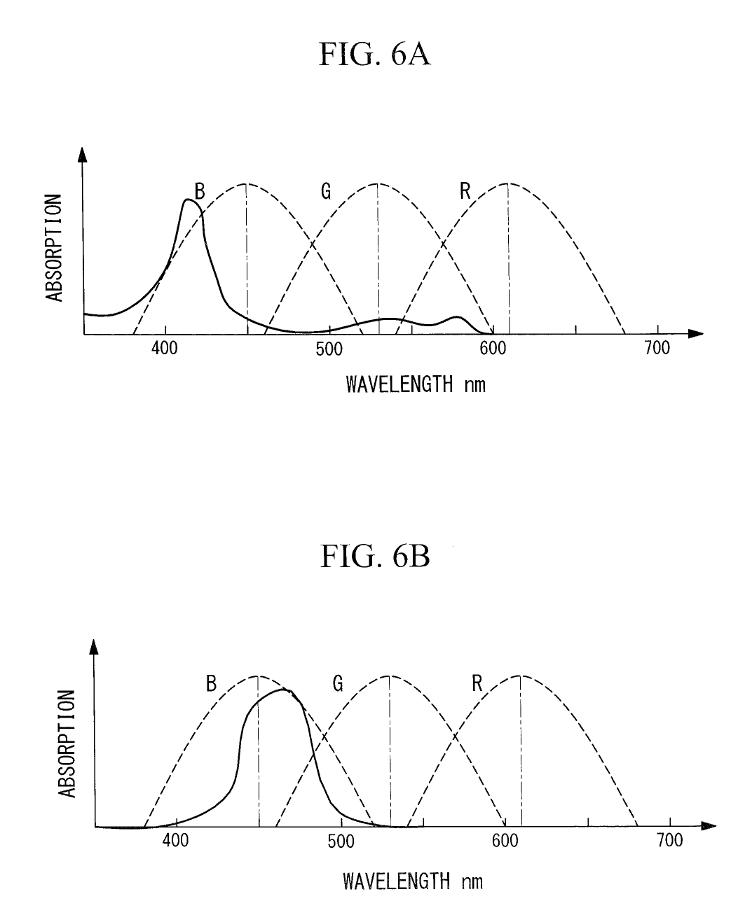

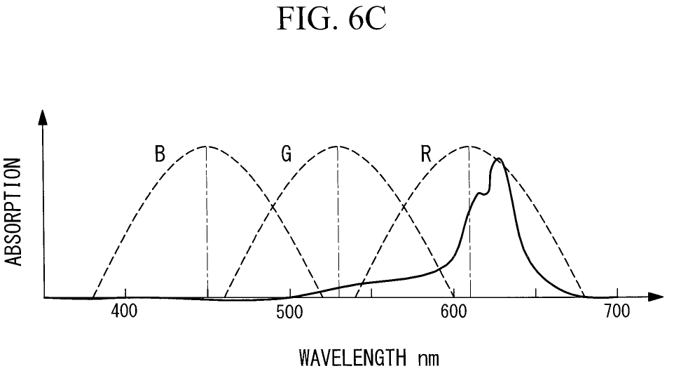

FIG. 6A is a diagram showing the absorption characteristics of hemoglobin contained in the biological tissue X. As shown in FIG. 6A, hemoglobin existing in blood strongly absorbs light of the B1 wavelength band in a surface layer of the biological tissue X, and strongly absorbs light of the B2 wavelength band in a deep layer of the biological tissue X.

Therefore, by constituting an image by using the image signals of R, G, and B1 wavelength bands, it is possible to generate an image in which the capillaries or the like in a surface layer of a living organism are emphasized. In addition, by constituting an image by using the image signals of R, G, and B2 wavelength bands, it is possible to generate an image in which a deep layer of the living organism is emphasized and in which the capillaries and light bleeding at the surface are not displayed.

The control portion 19 performs control so that the rotation of the filter turret 15 of the light-source portion 3 and image capturing by the image-acquisition device 12 are performed in a synchronized manner, so that the image signals acquired by the image-acquisition device 12 are stored in the memory 17, and so that the image-processing portion 18 generates any one of the above-described images on the basis of the image signals read out from the memory 17.

The operation of the thus-configured biological observation apparatus 1 according to this embodiment will be described below.

With the biological observation apparatus 1 according to this embodiment, white light generated by the xenon lamp 14 passes through one of the spectral filters F1 and F2 disposed in the optical path by the rotation of the filter turret 15, whereby two sets of narrow-band light are extracted, and the light is focused by the focusing lens 16 and is made to enter the light-guide cable 9 from the entrance end thereof.

The illumination light guided to the distal end 2a of the inserted portion 2 by the light-guide cable 9 is radiated onto the biological tissue X disposed so as to face the distal-end surface of the inserted portion 2, the light reflected at the biological tissue X forms an image by means of the lens 11, and the image is captured by the image-acquisition device 12.

Because the filters that individually transmit, for separate pixels, light in the R, G, and B wavelength bands are disposed in the image-acquisition device 12, of the light reflected at the biological tissue X, the reflected light of wavelength bands contained in the respective R, G, and B wavelength bands is captured at the pixels corresponding thereto.

In other words, when the first spectral filter F1, which transmits light in the R, G, and B1 wavelength bands, is disposed in the optical path, the image-acquisition device 12 captures the reflected light having the R, G, and B1 wavelength bands at pixels corresponding thereto, and thus, three types of image signals are acquired and stored in the memory 17. In addition, when the second spectral filter F2, which transmits light in the R, G, and B2 wavelength bands, is disposed in the optical path, the image-acquisition device 12 captures the reflected light having the R, G, and B2 wavelength bands at pixels corresponding thereto, and thus, three types of image signals are acquired and stored in the memory 17.

The control portion 19 causes the one set of image signals, which is formed of four types of image signals, stored in the memory 17 to be transmitted from the memory 17 to the image-processing portion 18. Then, the image-processing portion 18 generates a normal observation image constituted of all image signals and a special-light image constituted of the selected image signals, and the images are displayed on the monitor 5.

As has been described above, with the biological observation apparatus 1 according to this embodiment, there is an advantage in that it is possible to acquire a normal observation image and two types of special-light images just by disposing the two types of filters F1 and F2 in the optical path by switching between them. Therefore, it is not necessary to provide as many filters as the number of images to be observed, and thus, there is an advantage in that it is possible to perform observation at low cost by preventing increases in the size and the complexity of the apparatus.

In this embodiment, although the two narrow-band beams on either side of the central wavelength of the wavelength band are extracted from the light in the B wavelength band constituting the illumination light, alternatively, it is permissible to extract two narrow-band beams on either side of the central wavelength of the wavelength band from the light in the R or G wavelength band.

Next, a biological observation apparatus 22 according to a second embodiment of the present invention will be described below with reference to the drawings.

In the description of this embodiment, portions thereof having the same configurations as those of the biological observation apparatus 1 according to the first embodiment described above will be given the same reference signs, and descriptions thereof will be omitted.

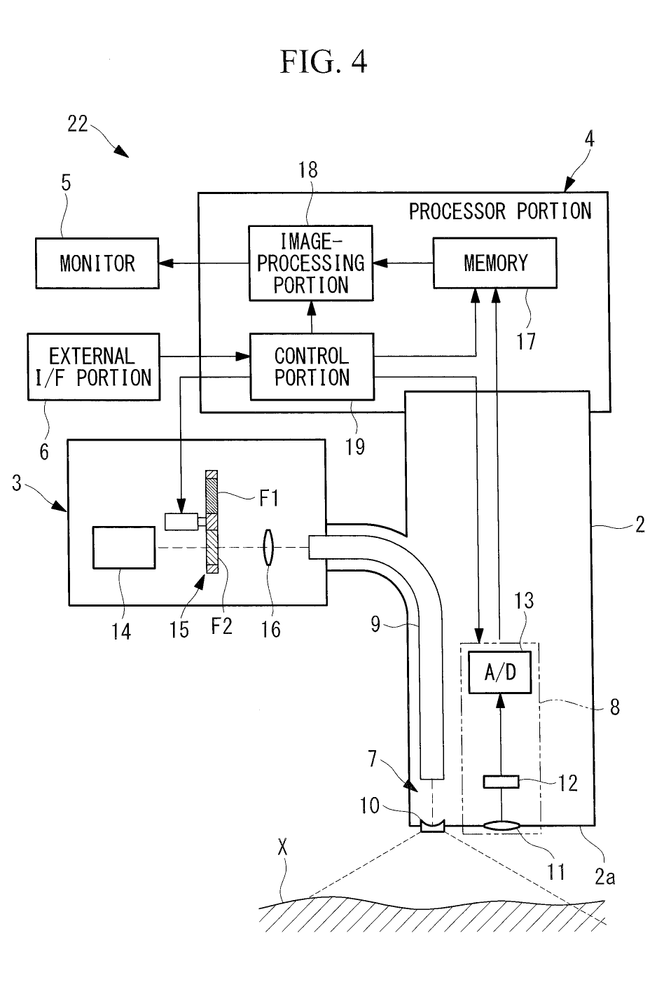

As shown in FIG. 4, the biological observation apparatus 22 according to this embodiment differs from the biological observation apparatus 1 according to the first embodiment in that the biological observation apparatus 22 is provided with an external I/F portion 6 with which an operator performs input operations to the processor portion 4, thus forming a narrow-band-light generating portion that extracts two narrow-band beams on either side of the central wavelength of the wavelength band from at least one of the beams in the R, G, and B wavelength bands constituting the illumination light.

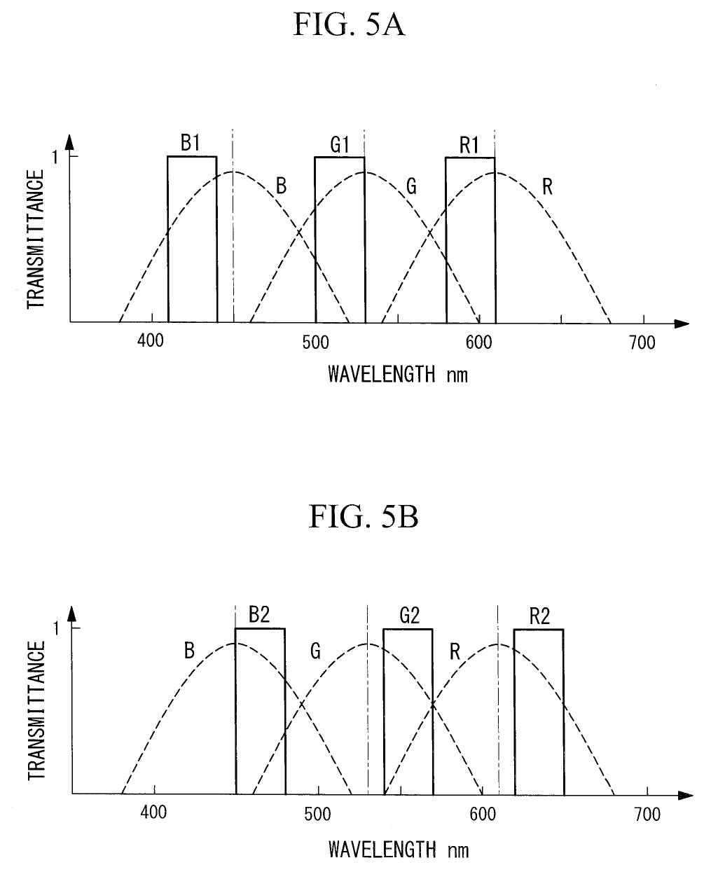

As shown in FIG. 5A, the first spectral filter F1 has B1 (410 to 440 nm), G1 (500 to 530 nm), and R1 (580 to 610 nm) transmission wavelength bands. In the figure, the broken line indicates the sensitivity of the color CCD 12, and the dashed lines individually indicate the central wavelengths of the R, G, and B wavelength bands.

As shown in FIG. 5B, the second spectral filter F2 has B2 (450 to 480 nm), G2 (540 to 570 nm), and R2 (620 to 650 nm) transmission wavelength bands. In the figure, the broken line indicates the sensitivity of the color CCD 12, and the dashed lines individually indicate the central wavelengths of the R, G, and B wavelength bands.

The transmission wavelength bands B1 and B2 belong to the B wavelength band constituting the white light and are arranged on either side of 450 nm, which is the central wavelength of the B wavelength band. The transmission wavelength bands G1 and G2 belong to the G wavelength band constituting the white light and are arranged on either side of 530 nm, which is the central wavelength of the G wavelength band. In addition, the transmission wavelength bands R1 and R2 belong to the R wavelength band constituting the white light and are arranged on either side of 610 nm, which is the central wavelength of the R wavelength band.

When the respective spectral filters F1 and F2 are disposed in the optical path, the wavelength characteristics of light individually captured at the R, G, and B pixels of the color CCD 12 are as shown in Table 2. As shown in Table 2, by using combinations of the two types of spectral filters F1 and F2 and the three types of pixels, that is, the R, G, and B pixels, it is possible to obtain image signals individually having different wavelength components. Therefore, six types of image signals are obtained. In other words, the two spectral filters F1 and F2 constitute a narrow-band-light generating portion that extracts, from the light of at least one of the R, G, and B wavelength bands constituting the illumination light, two narrow-band beams on either side of the central wavelength of the wavelength band.

TABLE 2

| | FIRST | SECOND |
|---|---|---|
| | SPECTRAL FILTER | SPECTRAL FILTER |
| B PIXEL | 410-440 nm (B1) | 450-480 nm (B2) |
| G PIXEL | 500-530 nm (G1) | 540-570 nm (G2) |
| R PIXEL | 580-610 nm (R1) | 620-650 nm (R2) |

The image-processing portion 18 is configured so as to generate images shown in Table 3 by using combinations of the image signals corresponding to the individual wavelengths in Table 2, stored in the memory 17.

TABLE 3

| | B | G | R |
|---|---|---|---|
| NORMAL OBSERVATION IMAGE | B1 + B2 | G1 + G2 | R1 + R2 |
| METHYLENE-BLUE | B1 + B2 | G1 + G2 | R2 |
| EMPHASIZED IMAGE | | | |
| FAT EMPHASIZED IMAGE | B2 | G1 + G2 | R1 + R2 |
| BLOOD EMPHASIZED IMAGE | B1 | G2 | R1 |

The individual images in Table 3 generated by the image-processing portion 18 will be described below in detail.

A normal observation image is an image that is constituted of all image signals of the R1, R2, G1, G2, B1, and B2 wavelength bands acquired by the image-acquisition device 12. The normal observation image is an image in which the B image signals are the sum of the B1 and B2 image signals, the G image signals are the sum of the G1 and G2 image signals, and the R image signals are the sum of the R1 and R2 image signals, respectively.

A blood emphasized image is a special-light image that is constituted of the image signals of the R1, G2, and B1 wavelength bands acquired by the image-acquisition device 12. FIG. 6A is a diagram showing the absorption characteristics of hemoglobin contained in the biological tissue X. As shown in FIG. 6A, the R1, G2, and B1 wavelength bands are wavelengths bands in which hemoglobin exhibits greater absorption than in the R2, G1, and B2 wavelength bands. Therefore, by constituting an image by using the image signals of these R1, G2, and B1 wavelength bands, it is possible to generate an image in which blood is emphasized. By constituting an image by selecting one each of the R1, G2, and B1 wavelength bands from all R, G, and B wavelength bands, it is possible to generate a well-balanced image that is easy to view.

Because the scattering characteristics in a living organism depend on the wavelength, short-wavelength light is scattered at a shallow position from the surface, and long-wavelength light is scattered at a deep position from the surface. Therefore, in the case in which it is necessary to emphasize only blood (blood vessel) at a surface layer, the B1 wavelength band, in which the absorption by hemoglobin is high, may be used in the B wavelength band, in which the wavelength thereof is short, and the G1 and R2 wavelength bands, in which the absorption by hemoglobin is low, may be used in the G and R wavelength bands.

A fat emphasized image is a special-light image that is constituted of the image signals of the R1, R2, G1, G2, and B2 wavelength bands acquired by the image-acquisition device 12. FIG. 6B is a diagram showing the absorption characteristics of β-carotene contained in the biological tissue X. As shown in FIG. 6B, the absorption by β-carotene, a large quantity of which is contained in fat, is notably high in the B2 wavelength band. Therefore, it is possible to generate an image in which β-carotene is emphasized by selecting only the image signals of the B2 wavelength band from the B wavelength band constituting the color image.

An exogenous-dye emphasized image is a special-light image in which an exogenous dye, such as methylene blue, Lugol's dye, or the like, that is used in endoscope examination to stain the living organism is emphasized instead of pigments existing in the living organism. For example, a methylene-blue emphasized image is an image that is constituted of the image signals of the R2, G1, G2, B1, and B2 wavelength bands acquired by the image-acquisition device 12. FIG. 6C is a diagram showing the absorption characteristics of methylene blue. As shown in FIG. 6C, the absorption by methylene blue is notably high in the R2 wavelength band. Therefore, it is possible to generate an image in which methylene blue is emphasized by selecting only the image signals of the R2 wavelength band from the R wavelength band constituting the color image.

The external I/F portion 6 is an input device, such as a keyboard or the like, that is operated by the operator, and with which it is possible to give inputs for selecting the special-light image to be generated by the image-processing portion 18.

The monitor 5 is configured so as to simultaneously display the normal observation image and one of the above-described special-light images, which are generated by the processor portion 4. In the case in which a special-light image is not obtained, only the normal observation image may be displayed. With regard to the special-light images, one of the above-described special-light images is selected by means of the selection made by the operator via the external I/F portion 6.

With the biological observation apparatus 22 according to this embodiment, when the first spectral filter F1, which transmits light in the R1, G1, and B1 wavelength bands, is disposed in the optical path, the image-acquisition device 12 captures the reflected light having the R1, G1, and B1 wavelength bands at pixels corresponding thereto, and thus, three types of image signals are acquired and stored in the memory 17. In addition, when the second spectral filter F2, which transmits light in the R2, G2, and B2 wavelength bands, is disposed in the optical path, the image-acquisition device 12 captures the reflected light having the R2, G2, and B2 wavelength bands at pixels corresponding thereto, and thus, three types of image signals are acquired and stored in the memory 17.

The control portion 19 causes the one set of image signals, which is formed of six types of image signals, stored in the memory 17 to be transmitted from the memory 17 to the image-processing portion 18. Then, the image-processing portion 18 generates a normal observation image in which all image signals are added up and a special-light image constituted of the image signals, the combination thereof is set based on the instruction input via the external I/F portion 6, and the images are displayed on the monitor 5. In addition, it is possible to generate and display different types of the special-light images by means of an input via the external I/F portion 6.

As has been described above, with the biological observation apparatus 1 according to this embodiment, there is an advantage in that it is possible to acquire a normal observation image and two or more types of special-light images just by disposing the two types of filters F1 and F2 in the optical path by switching between them.

With the biological observation apparatus 22 according to this embodiment, in the respective R, G, and B wavelength bands, two narrow-band beams on either side of the central wavelength of the wavelength band are extracted, and therefore, it is possible to select a wavelength band in which the absorption by the observation subject component contained in the living organism is high on one side thereof whereas the absorption is low on the other side thereof. By doing so, it is possible to perform high-contrast observation of the observation subject component by acquiring image signals by separately capturing reflected light of the two narrow bands.

With the biological observation apparatus 22 according to this embodiment, because a normal observation image and a special-light image are simultaneously displayed on the monitor 5, there is an advantage in that it is possible to perform observation by using the special-light image in which the observation subject component is emphasized while checking the state of the surface of the biological tissue X in the normal observation image that is constantly displayed and in which colors close to those of an image obtained during white-light illumination are reproduced.

With the biological observation apparatus 1 according to this embodiment, because a special-light image is generated on the basis of the image signals that are acquired by capturing reflected light in three types of narrow bands selected one each from the R, G, and B wavelength bands, with the special-light image also, it is possible to form an image in which the R, G, and B wavelength bands are well balanced and that is easy to view.

In the biological observation apparatus 22 according to this embodiment, although a special-light image is generated by using the instruction input by the operator via the external I/F portion 6, alternatively, the processing details (display content) may be set in advance, and a special-light image generated in accordance with the processing details may be displayed on the monitor 5. In this case, because the operator does not need to input the instruction via the external I/F portion 6, the external I/F portion 6 need not be provided.

Next, a biological observation apparatus according to a third embodiment of the present invention will be described below with reference to the drawings.

In the description of this embodiment, portions thereof having the same configurations as those of the biological observation apparatus 22 according to the second embodiment described above will be given the same reference signs, and descriptions thereof will be omitted.

The biological observation apparatus according to this embodiment differs from the biological observation apparatus 22 according to the second embodiment in that the spectral filters F1 and F2 are set so as to extract, from the respective R, G, and B wavelength bands, a first narrow band in which the absorption by the observation subject component (absorption characteristics) is the highest and a second narrow band that does not overlap with the first narrow band. With the thus-configured biological observation apparatus according to this embodiment, it is possible to perform high-contrast observation of the observation subject component by acquiring image signals by separately capturing reflected light in the two narrow bands.

An example in which hemoglobin is used as an observation subject component will be described.

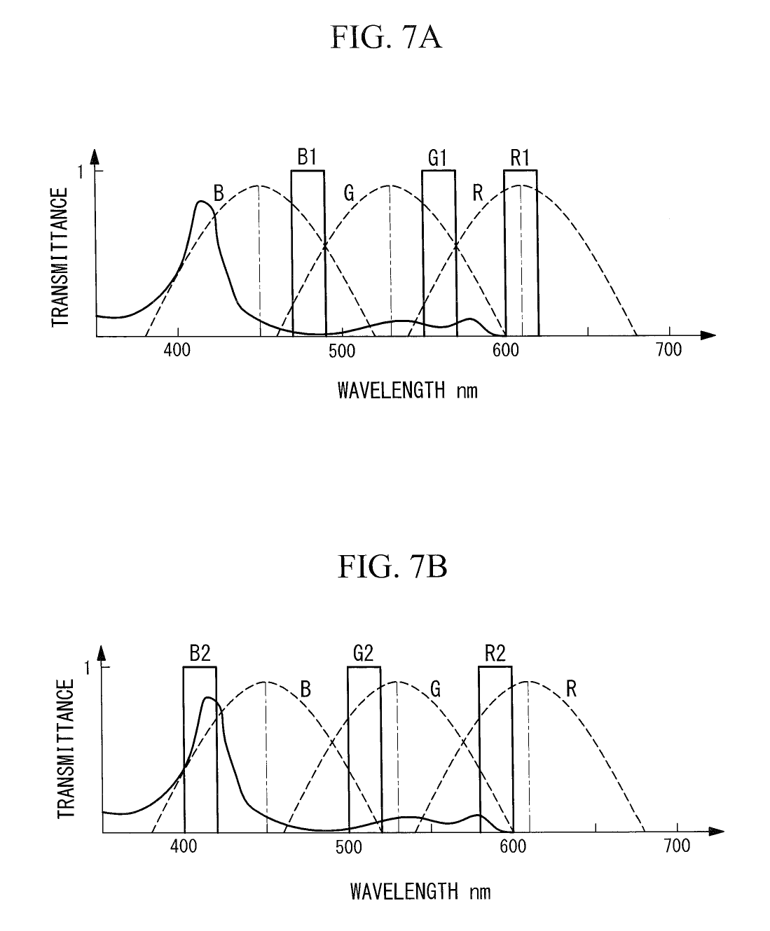

As shown in Table 4 and FIG. 7A, the first spectral filter F1 in this case has B1 (470 to 490 nm), G1 (550 to 570 nm), and R1 (600 to 620 nm) transmission wavelength bands. In addition, as shown in Table 4 and FIG. 7B, the second spectral filter F2 has B2 (400 to 420 nm), G2 (500 to 520 nm), and R2 (580 to 600 nm) transmission wavelength bands.

TABLE 4

| | FIRST | SECOND |
|---|---|---|
| | SPECTRAL FILTER | SPECTRAL FILTER |
| B PIXEL | 470-490 nm (B1) | 400-420 nm (B2) |
| G PIXEL | 550-570 nm (G1) | 500-520 nm (G2) |
| R PIXEL | 600-620 nm (R1) | 580-600 nm (R2) |

Thus, in this case, the image-processing portion 18 can generate images shown in Table 5 by using combinations of the image signals corresponding to the individual wavelengths in Table 4 stored in the memory 17.

TABLE 5

| | B | G | R |
|---|---|---|---|
| NORMAL OBSERVATION IMAGE | B1 + B2 | G1 + G2 | R1 + R2 |
| BLOOD EMPHASIZED IMAGE | B2 | G1 | R2 |
| BLOOD REDUCED IMAGE | B1 | G2 | R1 |
| INTERMEDIATE-PORTION BLOOD- | B1 | G1 | R1 |
| VESSEL EMPHASIZED IMAGE | | | |

A blood emphasized image is an image that is constituted of the image signals of the B2, G1, and R2 narrow bands in which the absorption by hemoglobin is high in the respective R, G, and B wavelength bands. By doing so, it is possible to display an image in which blood is emphasized.

A blood reduced image is a combined image that is constituted of the image signals of the B1, G2, and R1 narrow bands in which the absorption by hemoglobin is low in the respective R, G, and B wavelength bands. By doing so, it is possible to display an image in which the influence of blood is reduced.

With an intermediate-portion blood-vessel emphasized image, among the B2, G1, and R2 narrow bands of the blood emphasized image, light in the G1 narrow band is scattered at an intermediate depth in the living organism. Therefore, with respect to the B and R wavelength bands, by using the image signals of the B1 and R1 narrow bands, which do not emphasize blood, and by using the image signals of the G1 narrow band, which emphasizes blood, only for the G wavelength band, it is possible to display the blood vessels existing at the intermediate depth in an emphasized state.

In this embodiment, although two of each type of image signals acquired in the respective R, G, and B wavelength bands are used to separately constitute one type of image to be displayed, alternatively, two signals in the respective R, G, and B regions may be weighted and added up. For example, when adding up the image signals of the B1 and B2 wavelength bands in the B wavelength band, the operator may change the proportions of the B1 and B2 signals.

By doing so, it is possible to change the proportion of the influence of the observation subject component, for example, hemoglobin, in the image displayed on the monitor 5 in accordance with the surgical scene so as to facilitate the procedure.

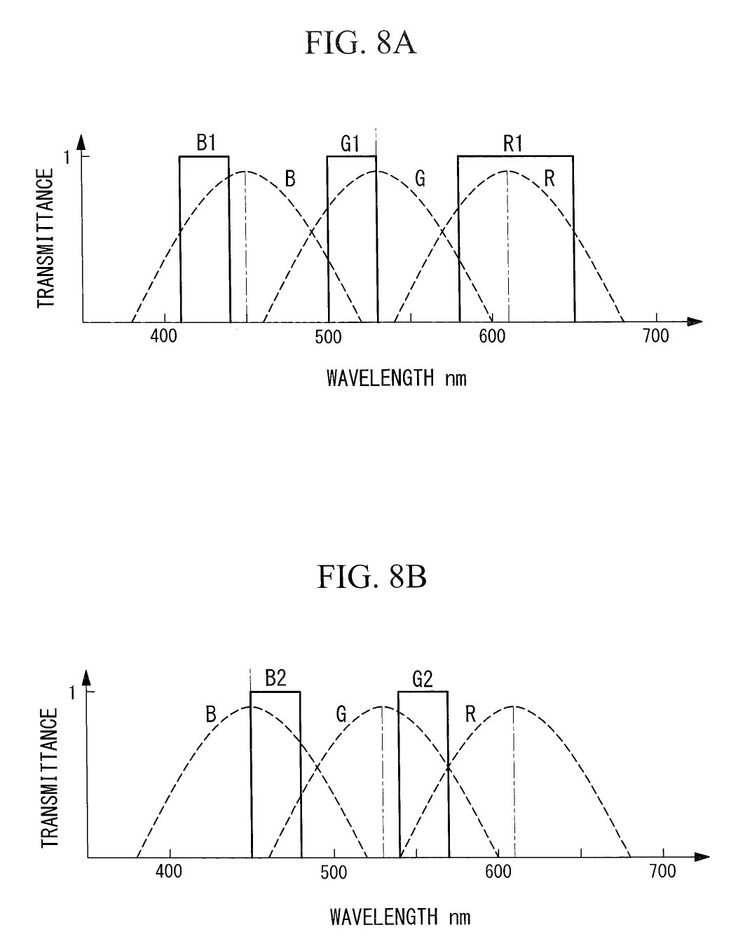

In this embodiment, although two of each type of image signals are acquired in all of the R, G, and B wavelength bands, alternatively, as shown in FIGS. 8A and 8B, two types of image signals may be acquired only for two wavelength bands, and, for the rest of the wavelength bands, image signals of wavelength bands on either side of the central wavelength may be acquired. FIGS. 8A and 8B show an example in which two types of transmission wavelength bands are provided for the B and G wavelength bands, and, regarding the R wavelength band, one type, that is, the R1 transmission wavelength band, is provided only in the first spectral filter F1.

Although the xenon lamp 14 has been described as an example of the light source, alternatively, another white light source, such as a halogen lamp, a mercury lamp, a white LED, or the like, may be employed.

In addition, although a normal observation image is generated by combining all of acquired image signals, for which there are two types in each of the R, G, and B wavelength bands, alternatively, in the case in which only normal observation is performed, both of the spectral filters F1 and F2 may be removed from the optical path, or a filter that transmits all light coming from the white light source may be disposed in the optical path.

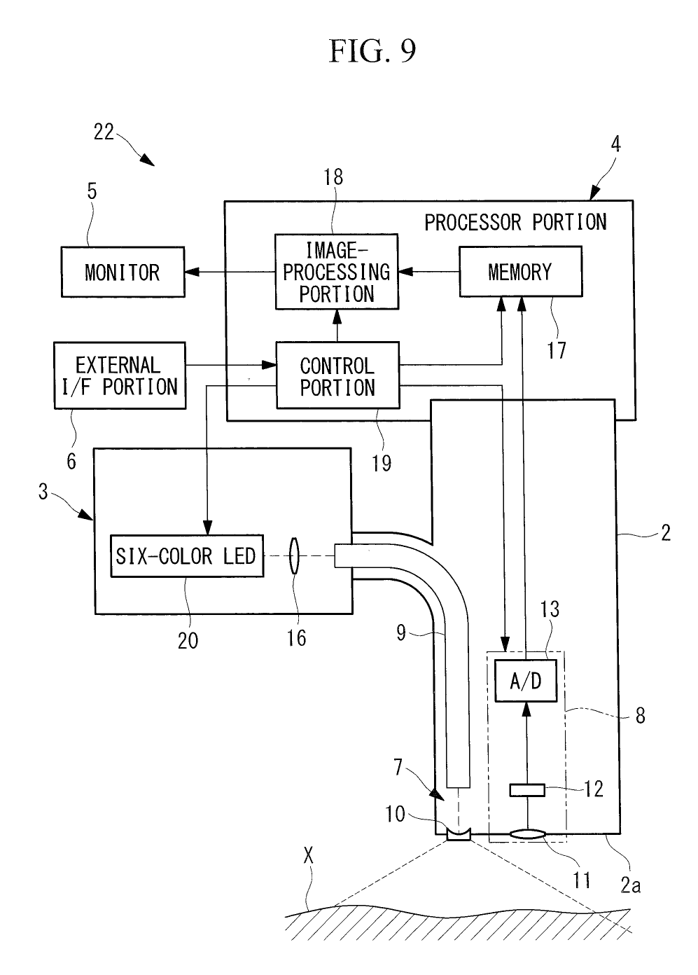

In this embodiment, although the light-source portion 3 generates two sets of narrow-band beams by using the xenon lamp 14 and the filter turret 15, alternatively, as shown in FIG. 9, the light-source portion 3 may be constituted of a six-color LED (illuminating portion and narrow-band-light generating portion) 20.

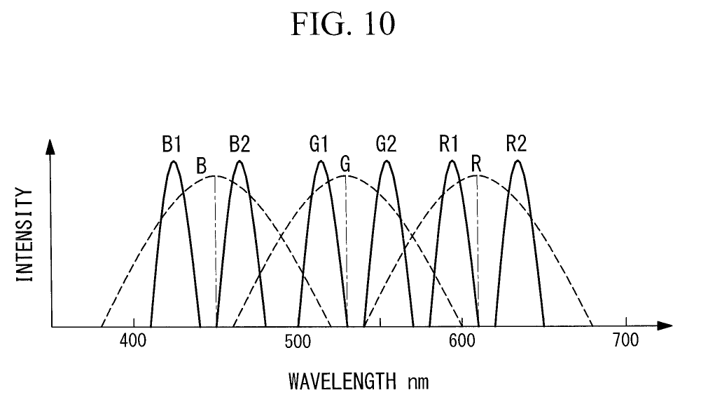

In this case, as shown in FIG. 10, first to sixth LEDs emit light corresponding to the B1, B2, G1, G2, R1, and R2 wavelength bands, wherein only the first, third, and fifth LEDs may be turned on at a first timing, only the second, fourth, and sixth LEDs may be turned on at a second timing, and this may be repeated in an alternating manner.

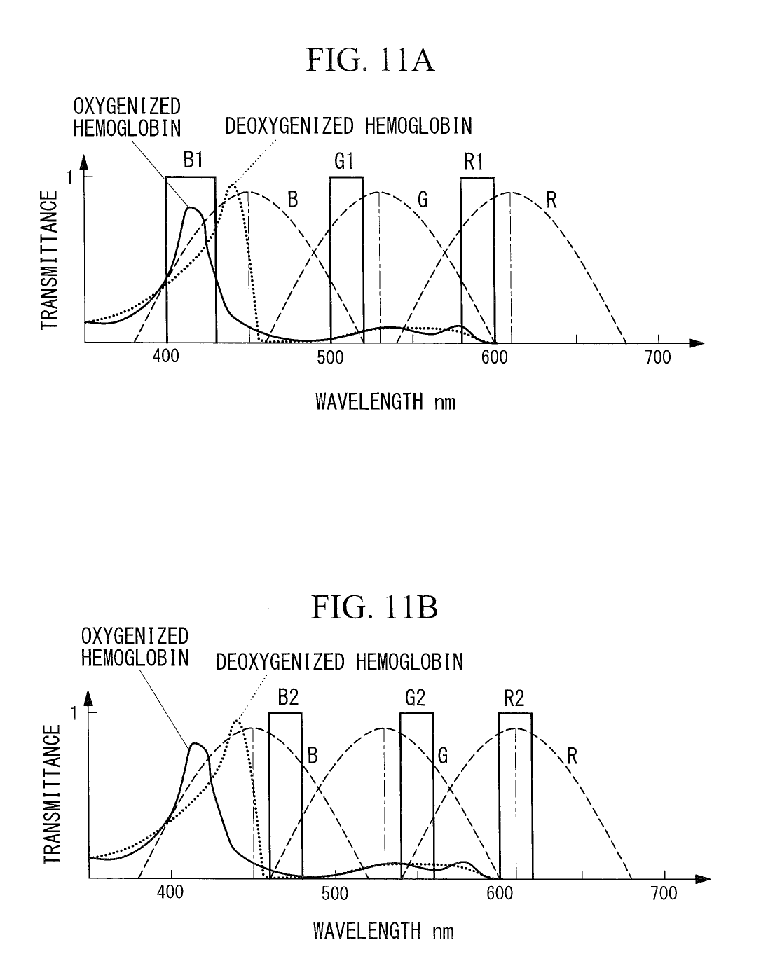

Although the amount of dye existing in the living organism has been described as an example of the observation subject component, alternatively, the oxygen saturation level may be used as the observation subject component. In this case, the spectral filters F1 and F2 having transmission wavelength bands shown in Table 6, FIGS. 11A and 11B are used. In addition, as shown in Table 7, the oxygen saturation level can be determined by calculating the ratio B2/G2 of the B2 and G2 narrow bands. The B2 narrow band is a wavelength band in which there is a concentration difference between oxygenized hemoglobin and deoxygenized hemoglobin, whereas the G2 narrow band G2 is a wavelength band in which there is no concentration difference between the two.

TABLE 6

| | FIRST SPECTRAL FILTER | SECOND SPECTRAL FILTER |
|---|---|---|
| B PIXEL | 400-430 nm (B1) | 460-480 nm (B2) |
| G PIXEL | 500-520 nm (G1) | 540-560 nm (G2) |
| R PIXEL | 580-600 nm (R1) | 600-620 nm (R2) |

TABLE 7

| | B | G | R |
|---|---|---|---|
| NORMAL OBSERVATION | B1 + B2 | G1 + G2 | R1 + R2 |
| IMAGE | | | |
| OXYGEN-SATURATION- | B2/G2 IS CALCULATED, AND | | |
| LEVEL IMAGE | COLOR IS APPLIED ON THE | | |
| | BASIS OF TABLE ASSOCIATING | | |
| | COLORS WITH B2/G2, WHICH IS | | |
| | STORED IN ADVANCE | | |
| BLOOD EMPHASIZED IMAGE | B1 | G2 | R1 |

By storing a table in which the ratios B2/G2 are associated with colors and by applying colors that are read out in accordance with the calculated ratios to image signals, it is possible to display a distribution of the oxygen saturation level in the form of color differences. The method of displaying the distribution of the oxygen saturation level is not limited thereto; by combining images by using image signals of the B2 narrow band as the image signals of the B wavelength band and image signals of the G2 narrow band as the image signals of the G wavelength band, a color distribution in which the balance of the B and G wavelength bands differs in accordance with the oxygen saturation level may be displayed.

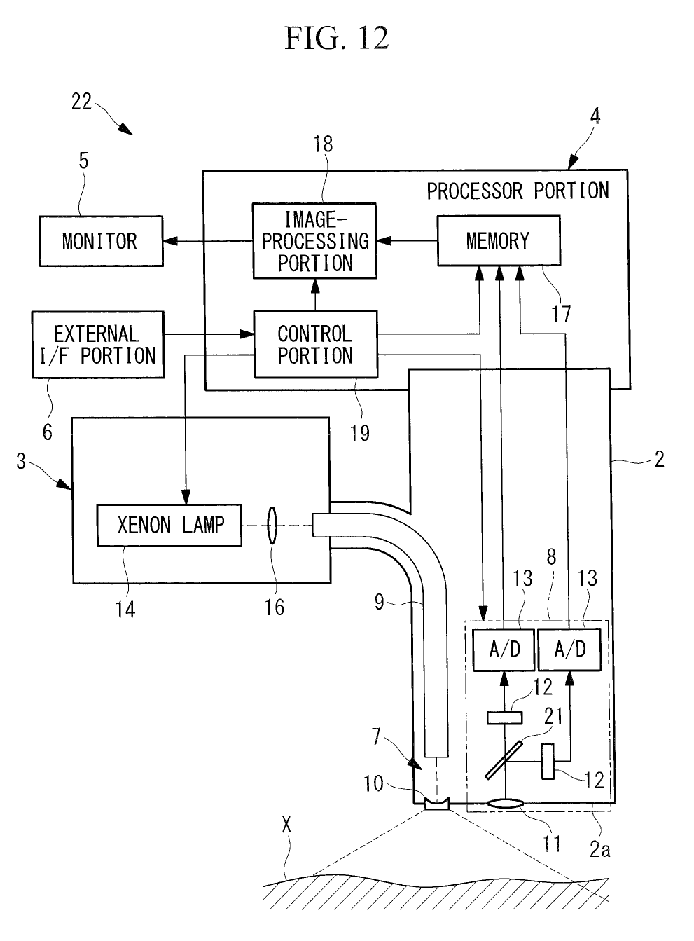

In the individual embodiments described above, although the case in which the light-source portion 3 is provided with a narrow-band-light generating portion that generates illumination light constituted of six types of narrow bands has been described, alternatively, as shown in FIG. 12, the white light emitted from the light-source portion 3 may be radiated onto the biological tissue X, and A beam splitter 21 may be disposed in the imaging optical system 8 so as to serve as the narrow-band-light generating portion.

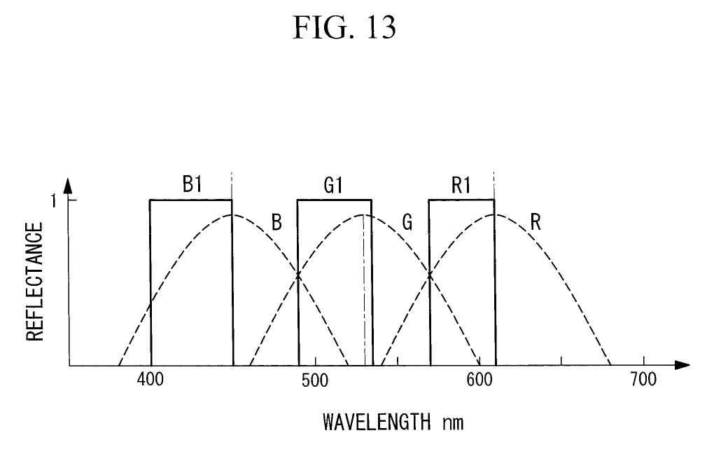

In other words, by disposing the beam splitter 21 having reflectance characteristics shown in FIG. 13 and by disposing the color CCD 12 and the A/D converter 13 on the reflecting side and the transmitting side of the beam splitter 21, respectively, it is possible to obtain six types of image signals acquired by capturing reflected light of six types of narrow bands shown in Table 8.

TABLE 8

| | REFLECTING- | TRANSMITTING- |
|---|---|---|
| | SIDE COLOR | SIDE COLOR |
| | CCD | CCD |
| B PIXEL | 400-450 nm (B1) | 450-490 nm (B2) |
| G PIXEL | 490-535 nm (G1) | 535-570 nm (G2) |
| R PIXEL | 570-610 nm (R1) | 610-650 nm (R2) |

As a result, the following aspect is read from the above described embodiment of the present invention.

An aspect of the present invention is a biological observation apparatus including: an illuminating portion that irradiates biological tissue with illumination light including light in R, G, and B regions, respectively; an image acquisition portion that acquires image signals from reflected light of the illumination light coming from the biological tissue; a narrow-band-light generating portion that is disposed in the illuminating portion or the image acquisition portion and that, in wavelength bands of the illumination light, generates two narrow-band beams for at least one of the R, G, and B wavelength bands constituting the illumination light, on either side of a central wavelength of that wavelength band; and an image-generating portion that generates an image on the basis of two or more types of the image signals obtained from the reflected light including two or more narrow bands acquired by the image acquisition portion.

With this aspect, when the illumination light emitted from the illuminating portion is radiated onto the biological tissue, the reflected light of the illumination light coming from the biological tissue is captured by the image acquisition portion, and thus the image signals are acquired. From the illumination light emitted from the illuminating portion or the reflected light coming from the biological tissue, the narrow-band-light generating portion generates two narrow-band beams from light in at least one of the R, G, and B wavelength bands.

In the case in which the narrow-band-light generating portion is disposed in the illuminating portion, the generated narrow-band beam is radiated onto the biological tissue, and reflected light of that narrow band is captured by the image acquisition portion. In the case in which the narrow-band-light generating portion is disposed in the image acquisition portion, the narrow-band beam is generated from the reflected light coming from the biological tissue and is captured by the image acquisition portion. In both cases, the reflected light of two or more narrow bands generated by the narrow-band-light generating portion is captured by the image acquisition portion, and the image-generating portion generates an image on the basis of the two or more acquired image signals.

The two narrow-band beams generated by the narrow-band-light generating portion are narrow-band beams on either side of the central wavelength of at least one of the R, G, and B wavelength bands, and, by combining the reflected light of the two narrow bands, it is possible to achieve well-balanced reproduction of light of the R, G or B wavelength band. By combining the reflected light of the two narrow bands for all of the R, G, and B wavelength bands, it is possible to perform observation by using an image in which colors close to those of an image obtained during white-light illumination are reproduced.

Another aspect of the present invention is a biological observation apparatus including: an illuminating portion that irradiates biological tissue with illumination light including light in R, G, and B regions, respectively; an image acquisition portion that acquires image signals from reflected light of the illumination light coming from the biological tissue; a narrow-band-light generating portion that is disposed in the illuminating portion or the image acquisition portion and that, in the wavelength band of the illumination light, generates light in a first narrow band including a wavelength at which absorption characteristics of an observation subject component reach a maximum and light in a second narrow band that is different from the first narrow band for at least one of the R, G, and B wavelength bands constituting the illumination light; and an image-generating portion that generates an image on the basis of two or more types of the image signals obtained from the reflected light including two or more narrow bands acquired by the image acquisition portion.

With this aspect, when the illumination light emitted from the illuminating portion is radiated onto the biological tissue, the reflected light of the illumination light coming from the biological tissue is captured by the image acquisition portion, and thus the image signals are acquired. From the illumination light emitted from the illuminating portion or the reflected light coming from the biological tissue, the narrow-band-light generating portion generates the first narrow-band beam and the second narrow-band beam from light of at least one of the R, G, and B wavelength bands.

In the case in which the narrow-band-light generating portion is disposed in the illuminating portion, the generated narrow-band beam is radiated onto the biological tissue, and reflected light of that narrow band is captured by the image acquisition portion. In the case in which the narrow-band-light generating portion is disposed in the image acquisition portion, the narrow-band beam is generated from the reflected light coming from the biological tissue and is captured by the image acquisition portion. In both cases, the reflected light of two or more narrow bands generated by the narrow-band-light generating portion is captured by the image acquisition portion, and the image-generating portion generates an image on the basis of the two or more acquired image signals.

The two narrow-band beams generated by the narrow-band-light generating portion are light of the first narrow band including the wavelength in which the absorption characteristics of the observation subject component reach the maximum in at least one of the R, G, and B wavelength bands and light of the second narrow band that is different from the first narrow band. By combining the reflected light of the two narrow bands, it is possible to achieve well-balanced reproduction of light of the R, G or B wavelength band, as compared with the case in which reflected light of one narrow band is used. By combining the reflected light of the two narrow bands for all of the R, G, and B wavelength bands, it is possible to perform observation by using an image in which colors close to those of an image obtained during white-light illumination are reproduced.

In the above-described aspect, the observation subject component may be β-carotene or hemoglobin.

By doing so, it is possible to observe fat or blood, which is contained in the biological tissue in a large quantity, in a precise manner.

In addition, in the above-described aspect, the image-generating portion may generate a plurality of images including a normal observation image in which the image signals acquired by the image acquisition portion, which are obtained from the reflected light including all narrow bands generated by the narrow-band-light generating portion, are used in combinations and a display portion that simultaneously displays the plurality of images including the normal observation image may be provided.

By doing so, by using combinations of the image signals acquired by capturing the reflected light of portions of the narrow bands, a special-light image, with which it is possible to observe a specific observation subject component with high contrast, is generated, and a normal observation image, in which the image signals acquired by capturing the reflected light of all of the narrow bands generated by the narrow-band-light generating portion are used in combinations, is generated. By simultaneously displaying the plurality of generated images on the display portion, it is possible to observe the observation subject component by using the special-light image, while constantly observing the external appearance of the biological tissue by using the normal observation image in which colors close to those of an image obtained during white-light illumination are reproduced.

### REFERENCE SIGNS LIST

1, 22 biological observation apparatus
3 light-source portion (illuminating portion)
5 monitor (display portion)
7 illumination optical system (illuminating portion)
8 imaging optical system (image acquisition portion)
18 image-processing portion (image-generating portion)
21 beam splitter (narrow-band-light generating portion)
F1, F2 spectral filter (narrow-band-light generating portion)
X biological tissue

1. A biological observation apparatus comprising:
an illuminating portion that irradiates biological tissue with illumination light including light in R, G, and B regions, respectively;
an image acquisition portion that acquires image signals from reflected light of the illumination light coming from the biological tissue;
a narrow-band-light generating portion that is disposed in the illuminating portion or the image acquisition portion and that, in wavelength bands of the illumination light, generates two narrow-band beams for at least one of the R, G, and B wavelength bands constituting the illumination light, on either side of a central wavelength of that wavelength band; and
an image-generating portion that generates an image on the basis of two or more types of the image signals obtained from the reflected light including two or more narrow bands acquired by the image acquisition portion.

2. A biological observation apparatus comprising:
an illuminating portion that irradiates biological tissue with illumination light including light in R, G, and B regions, respectively;
an image acquisition portion that acquires image signals from reflected light of the illumination light coming from the biological tissue;
a narrow-band-light generating portion that is disposed in the illuminating portion or the image acquisition portion and that, in the wavelength bands of the illumination light, generates light in a first narrow band including a wavelength at which absorption characteristics of an observation subject component reach a maximum and light in a second narrow band that is different from the first narrow band for at least one of the R, G, and B wavelength bands constituting the illumination light; and
an image-generating portion that generates an image on the basis of two or more types of the image signals obtained from the reflected light including two or more narrow bands acquired by the image acquisition portion.

3. A biological observation apparatus according to claim 2, wherein the observation subject component is β-carotene or hemoglobin.

4. A biological observation apparatus according to claim 1,
wherein the image-generating portion generates a plurality of images including a normal observation image in which the image signals acquired by the image acquisition portion, which are obtained from the reflected light including all narrow bands generated by the narrow-band-light generating portion, are used in combinations and
a display portion that simultaneously displays the plurality of images including the normal observation image is provided.

5. A biological observation apparatus according to claim 2,
wherein the image-generating portion generates a plurality of images including a normal observation image in which the image signals acquired by the image acquisition portion, which are obtained from the reflected light including all narrow bands generated by the narrow-band-light generating portion, are used in combinations and
a display portion that simultaneously displays the plurality of images including the normal observation image is provided.
